(19) United States
(12) Patent Application Publication
(10) Pub. No.: US 2023/0018783 A1
(43) Pub. Date: Jan. 19, 2023

(54) ENDOSCOPE OPERATION PORTION AND ENDOSCOPE

(71) Applicant: OLYMPUS CORPORATION, Tokyo (JP)

(72) Inventor: Yui KAWANISHI, Tokyo (JP)

(73) Assignee: OLYMPUS CORPORATION, Tokyo (JP)

(21) Appl. No.: 17/953,422

(22) Filed: Sep. 27, 2022

Related U.S. Application Data
(63) Continuation of application No. PCT/JP2020/014212, filed on Mar. 27, 2020.

(51) Int. Cl. A61B 1/005; A61B 1/00

(52) U.S. Cl. CPC A61B 1/0052; A61B 1/00042

(57) ABSTRACT

In an endoscope operation portion, a first finger placement portion and a second finger placement portion are disposed at positions where at least a pressing force of a first finger placed on the first finger placement portion, a pressing force of a second finger placed on the second finger placement portion, and an operation force amount of a third finger placed on a third finger placement portion when operating a bending operation lever are balanced.

### CROSS REFERENCE TO RELATED APPLICATION

This application is a continuation application of PCT/JP2020/014212 filed on Mar. 27, 2020, the entire contents of which are incorporated herein by this reference.

### BACKGROUND OF THE INVENTION

### 1. Field of the Invention

The present invention relates to an endoscope operation portion including a bending operation lever for operating a bending portion provided in an insertion portion, and an endoscope.

### 2. Description of the Related Art

As is well known, endoscopes are widely used for observation, treatment, and the like in the body (in body cavity) of a living body, or inspection, repair, and the like in an industrial plant facility. Such an endoscope includes an elongated insertion portion to be inserted into the body cavity, an abdominal cavity, a duct, and the like of a subject.

The insertion portion of the endoscope which is provided with a bending portion at a distal end portion thereof in order to improve insertability into a subject to be examined is known.

For example, as disclosed in International Publication No. WO2015/153960, a bending amount of the bending portion provided in the insertion portion is variably operated in a predetermined direction in accordance with an operation amount of a bending operation lever provided in a handle assembly as an operation portion.

### SUMMARY OF THE INVENTION

An endoscope operation portion according to an aspect of the present invention includes: a grasping portion including a distal end connected to an elongated insertion portion having a bending portion that can be bent, the grasping portion having a longitudinal axis and being configured to be grasped by a palm of an operator; a bending operation portion provided at a proximal end of the grasping portion and rotatably provided with a bending operation lever configured to bend the bending portion; a first finger placement portion formed in the bending operation portion on one surface side of the operation portion and having a recessed shape configured to be placed with a first finger of the operator; a second finger placement portion formed in the grasping portion on the one surface side of the operation portion and having a recessed shape configured to be placed with a second finger of the operator; and a third finger placement portion provided on the bending operation lever arranged on another surface side opposite to the one surface side of the operation portion configured to be placed with a third finger of the operator. The first finger placement portion and the second finger placement portion are disposed at positions where at least a pressing force of the first finger placed on the first finger placement portion, a pressing force of the second finger placed on the second finger placement portion, and an operation force amount of the third finger placed on the third finger placement portion at the time of operating the bending operation lever are balanced.

An endoscope according to an aspect of the present invention includes an endoscope operation portion, the endoscope operation portion including: a grasping portion including a distal end connected to an elongated insertion portion having a bending portion that can be bent, the grasping portion having a longitudinal axis and being configured to be grasped by a palm of an operator; a bending operation portion provided at a proximal end of the grasping portion and rotatably provided with a bending operation lever configured to bend the bending portion; a first finger placement portion formed in the bending operation portion on one surface side of the operation portion and having a recessed shape configured to be placed with a first finger of the operator; a second finger placement portion formed in the grasping portion on the one surface side of the operation portion and having a recessed shape configured to be placed with a second finger of the operator; and a third finger placement portion provided on the bending operation lever arranged on another surface side opposite to the one surface side of the operation portion configured to be placed with a third finger of the operator. The first finger placement portion and the second finger placement portion are disposed at positions where at least a pressing force of the first finger placed on the first finger placement portion, a pressing force of the second finger placed on the second finger placement portion, and an operation force amount of the third finger placed on the third finger placement portion at the time of operating the bending operation lever are balanced.

An endoscope according to another aspect of the present invention includes: a bending portion; an operation portion configured to be grasped by an operator to operate the bending portion; a bending operation lever provided freely movable in back and forth directions on a proximal end side of the operation portion and is configured to bend the bending portion; a first finger placement portion having a recessed shape provided on a surface side opposite to a surface side provided with the bending operation lever of the operation portion and formed with a surface facing a distal end side configured to be placed with a first finger of the operator; and a second finger placement portion having a recessed shape provided on a distal end side with respect to the first finger placement portion and formed with a surface facing a proximal end side configured to be placed with a second finger of the operator. A third finger placement portion is provided on the bending operation lever for placing a third finger of the operator, and the first finger placement portion and the second finger placement portion are disposed at positions where at least a pressing force of the first finger placed on the first finger placement portion, a pressing force of the second finger placed on the second finger placement portion, and an operation force amount of the third finger placed on the third finger placement portion at the time of operating the bending operation lever are balanced.

### BRIEF DESCRIPTION OF THE DRAWINGS

FIG. 1 is a perspective view illustrating an appearance of an endoscope according to an aspect of the present invention.

FIG. 2 is a front view illustrating an appearance of the endoscope according to an aspect of the present invention.

FIG. 3 is a top view illustrating an appearance of the endoscope according to an aspect of the present invention.

FIG. 4 is a right side view illustrating an appearance of the endoscope according to an aspect of the present invention.

FIG. 5 is a left side view illustrating an appearance of the endoscope according to an aspect of the present invention.

FIG. 6 is a plan view illustrating a state in which an operation portion is grasped by a user and a bending operation lever in a state in which a bending portion is maximally bent in one direction is rotationally operated according to an aspect of the present invention.

FIG. 7 is a plan view illustrating a state in which the operation portion is grasped by the user, and the bending operation lever in a state in which the bending portion is maximally bent in another direction is rotationally operated according to an aspect of the present invention.

### DETAILED DESCRIPTION OF THE PREFERRED EMBODIMENT(S)

Hereinafter, an endoscope and an endoscope operation portion according to an aspect of the present invention will be described with reference to the accompanying drawings. In the following description, the drawings based on the respective embodiments are schematic, and it should be noted that the relationship between a thickness and a width of each portion, a ratio of the thicknesses of the respective portions, and the like are different from the actual configuration. There may be cases where the relationship and the ratio of dimensions included in the drawings may be different among the different drawings.

The endoscope in the following description of the configuration is exemplified by a so-called flexible endoscope having a flexible insertion portion to be inserted into the stomach, the small intestine, the bile duct, the pancreatic duct, the large intestine, and the like in a body cavity from the bronchus, the urinary organ, or the esophagus of a living body. However, the endoscope of the present invention is not limited to the above, and the endoscope of the present invention is applicable to a so-called rigid endoscope in which the insertion portion having a bending portion for surgical use is rigid.

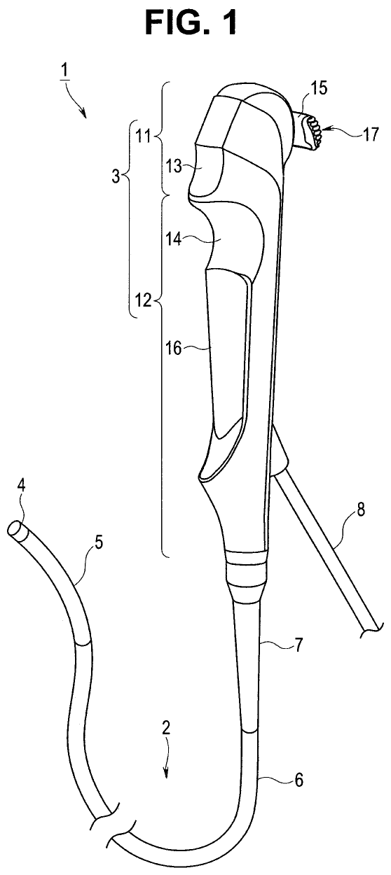

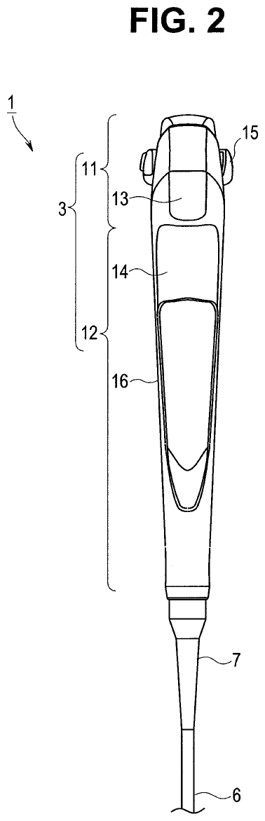

As illustrated in FIGS. 1 and 2, an endoscope 1 of the present embodiment is mainly configured with an elongated insertion portion 2 formed in an elongated tubular shape to be inserted into a subject, an operation portion 3 to which a proximal end of the insertion portion 2 is connected, a universal cord 8 which is an endoscope cable extending from a distal end side of the operation portion 3, an endoscope connector (not illustrated) provided at an extending end portion of the universal cord 8, and the like.

The insertion portion 2 is a flexible tubular member and formed by being consecutively connected to a distal end portion 4, a bending portion 5, and a flexible tube portion 6, in this order from the distal end side. In the distal end portion 4, an image pickup unit which is an image pickup apparatus including image pickup means therein, illumination means, and the like are housed and arranged (none are illustrated).

The bending portion 5 is a mechanism portion configured to be actively bent in two directions of up and down (UP-DOWN) with respect to a field of view of an image pickup unit (not illustrated) by a rotational operation of a bending operation lever 15 to be described later which is one of operation means of the operation portion 3.

Note that the bending portion 5 is not limited to a two-direction bending type, and may be a type that can be bent in four directions such as right and left directions in addition to the up and down directions with respect to the field of view of the image pickup unit (all circumferential directions around an axis, UP-DOWN/RIGHT-LEFT, by up-and-down and right-and-left operations).

The flexible tube portion 6 is a tubular member formed to have flexibility so as to be passively flexible. Various signal lines extending from the image pickup unit incorporated in the distal end portion 4 and further extending to the inside of the universal cord 8 via the operation portion 3, a light guide for guiding illumination light from a light source apparatus and emitting the illumination light from an illumination window disposed in the distal end portion 4, and the like are inserted through the inside of the flexible tube portion 6 (here, none are illustrated).

The operation portion 3 includes a grasping portion 12 which is provided at a distal end of the insertion portion 2 side and is grasped by a hand when a user uses the endoscope 1, and a bending operation portion 11 which is integrally and consecutively connected to a proximal end side of the grasping portion 12. The grasping portion 12 includes an operation portion main body 16 as a portion with which the palm of the user who is an operator comes into contact.

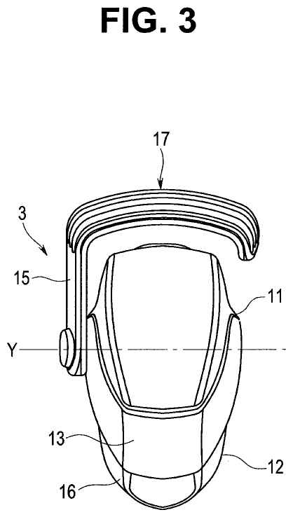

The bending operation lever 15 is provided on the bending operation portion 11. The bending operation lever 15 has a substantially L-shape and is rotationally operated around a rotation axis Y (see FIG. 3) to bend the bending portion 5 of the insertion portion 2 in two directions of up and down. In other words, the bending operation lever 15 is operated so as to move back and forth with respect to the grasping portion 12 on a back surface side of the bending operation portion 11 along a central axis X of the operation portion 3.

In addition, the operation portion 3 includes a bend preventing portion 7 that covers the proximal end of the flexible tube portion 6 of the insertion portion 2 and connects the flexible tube portion 6 and a distal end of the operation portion main body 16 on a distal end side of the grasping portion 12.

The universal cord 8 is provided to extend in an oblique direction toward the distal end side so as to have a predetermined angle with respect to the insertion portion 2 from the vicinity of the distal end on a back surface side of the operation portion main body 16 of the grasping portion 12.

In the universal cord 8, various signal lines are inserted from the distal end portion 4 of the insertion portion 2 to reach the grasping portion 12 and disposed further from the grasping portion 12 through inside the insertion portion 2. The universal cord 8 is a composite cable in which a light guide for transmitting light from a light source apparatus (not illustrated) is inserted therethrough.

Note that the endoscope connector provided at an extending end of the universal cord 8 is configured to include an electric connector portion to which a signal cable for detachably connecting to a video processor of an external device is connected on a side surface portion, a light source connector portion for detachably connecting to a light source apparatus which is an external device, and the like (both are not illustrated).

Here, with regard to the operation portion 3 of the endoscope 1, a configuration of main portions of the present embodiment will be described in detail below.

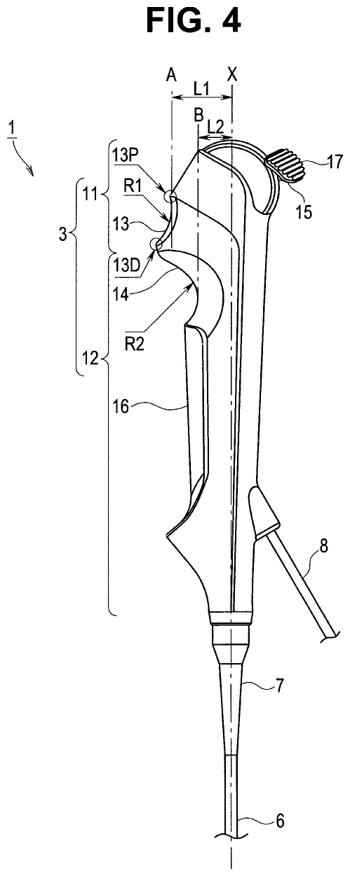

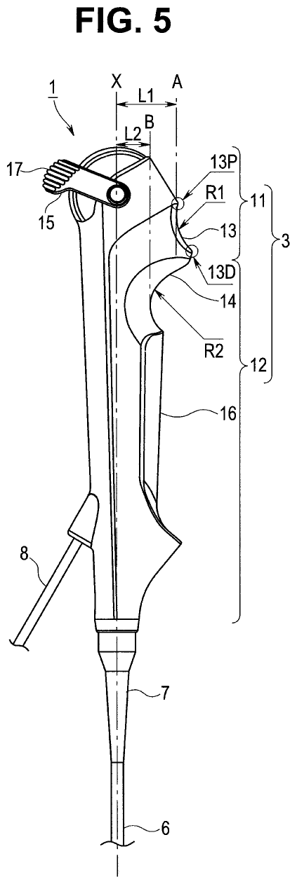

As illustrated in FIGS. 4 and 5, in the operation portion 3 of the endoscope 1, two finger placement portions 13 and 14 (first finger placement portion and second finger placement portion) having a recessed shape are formed on the front surface side. In addition, a finger placement portion 17 (third finger placement portion) having surface irregularities is formed on the bending operation lever 15 provided on a back surface side of the operation portion 3 as a direction confronting the user.

The first finger placement portion 13 is provided on the front surface side of a distal end portion of the bending operation portion 11 of the operation portion 3, and the second finger placement portion 14 is provided on the front surface side of a proximal end portion of the grasping portion 12. As described above, the first finger placement portion 13 and the second finger placement portion 14 are arranged side by side in proximity to each other in a direction along the central axis X which is a longitudinal axis connecting the distal end and the proximal end of the operation portion 3.

The first finger placement portion 13 has a recessed shape having a substantially arc-shaped cross section with a first curvature R1. The second finger placement portion 14 has a recessed shape having a substantially arc-shaped cross section with a second curvature R2 larger than the first curvature R1 of the first finger placement portion 13.

The respective recessed shapes of the first finger placement portion 13 and the second finger placement portion 14 are set such that the first finger placement portion 13 includes an arc-shaped surface facing the distal end side of the operation portion 3, and the second finger placement portion 14 includes an arc-shaped surface facing a proximal end side of the operation portion 3.

The depth of the recessed portion (bottom of recessed portion) is set on an axis A on which the first finger placement portion 13 is separated from the central axis X of the operation portion 3 by a predetermined distance L1, and the depth of the recessed portion (bottom of recessed portion) is set on an axis B on which the second finger placement portion 14 is separated from the central axis X of the operation portion 3 by a predetermined distance L2 shorter than the predetermined distance L1 (L1>L2).

In other words, the first finger placement portion 13 of the bending operation portion 11 is formed at a position farther from the central axis X of the operation portion 3 than the second finger placement portion 14 of the grasping portion 12. In addition, a position of an end 13D on the distal end side of the first finger placement portion 13 is formed at a position separated to a front surface side from an end 13P on a proximal end side of the first finger placement portion 13 with the central axis X as a reference.

In other words, the depth of the recessed portion of the first finger placement portion 13 is set to be shallow, and the first finger placement portion 13 is formed at a position closer to a front surface side of the operation portion 3 than the second finger placement portion 14. The depth of the recessed portion of the second finger placement portion 14 is set to be deep, and the second finger placement portion 14 is formed at a position closer to the central axis X of the operation portion 3 facing the back surface side of the operation portion 3 than the first finger placement portion 13.

In the endoscope 1 configured as described above, an action when a bending operation is performed on the bending portion 5 by the user will be described below.

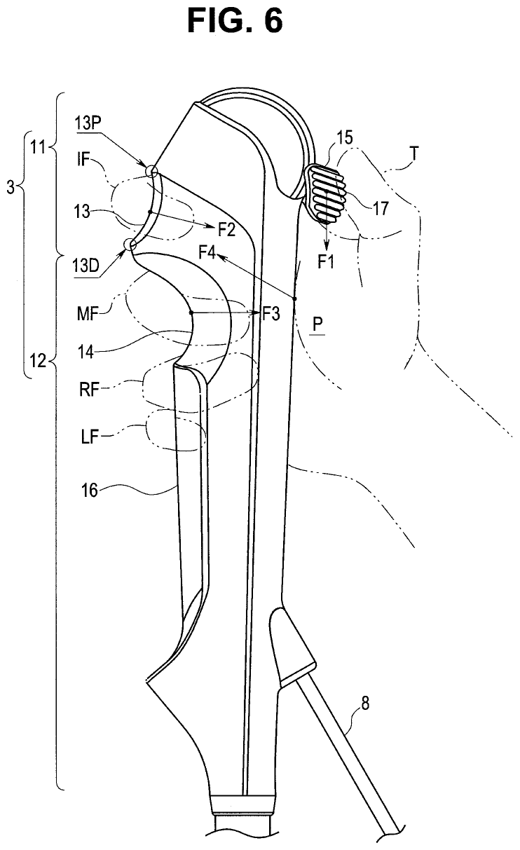

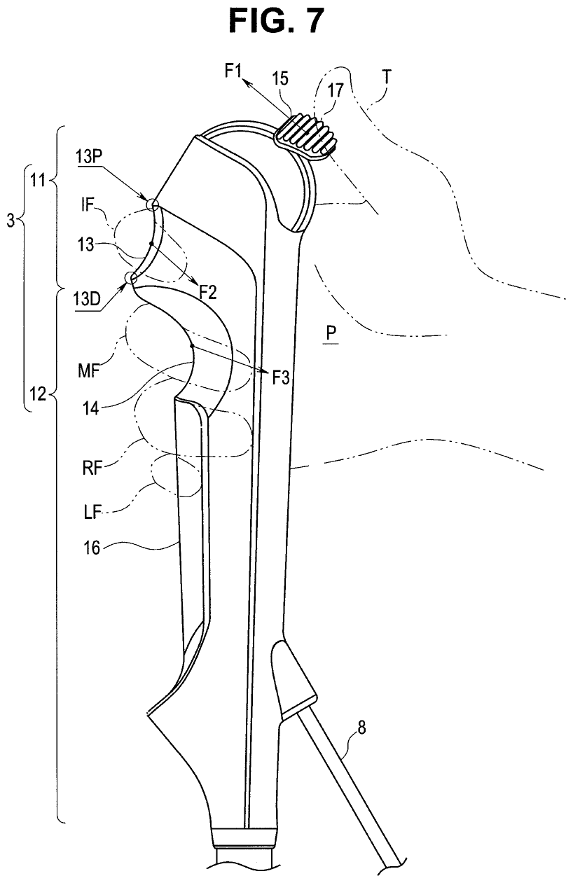

As illustrated in FIGS. 6 and 7, for example, the operation portion 3 is grasped by the user with an index finger IF placed on the first finger placement portion 13 on a front surface side of the bending operation portion 11, a middle finger MF placed on the second finger placement portion 14 on a front surface side of the grasping portion 12, a ring finger RF and a little finger LF placed on a front surface side of the operation portion main body 16 on the distal end side of the second finger placement portion 14, and a thumb T placed on the third finger placement portion 17 of the bending operation lever 15 on a back surface side of the grasping portion 12.

Note that the state illustrated in FIG. 6 illustrates a state in which the bending operation lever 15 is rotationally operated by the thumb T to one direction side (lower side which is distal end side when viewed toward paper surface), and the bending portion 5 is maximally bent in one predetermined direction, for example, in an UP direction. On the other hand, the state illustrated in FIG. 7 illustrates a state in which the bending operation lever 15 is rotationally operated by the thumb T to the other direction side (upper side when viewed toward the paper surface), and the bending portion 5 is maximally bent in predetermined another direction, for example, in a DOWN direction.

Here, a configuration in which the bending portion 5 is bent in the UP direction by rotationally operating the bending operation lever 15 in one direction and the bending portion 5 is bent in the DOWN direction by rotationally operating the bending operation lever 15 in the other direction has been described, but an UP-DOWN bending direction of the bending portion 5 with respect to the rotational operation direction of the bending operation lever 15 may be reversed.

First, in the state illustrated in FIG. 6, the operation portion 3 is in the state in which an operation position of the bending operation lever 15 is held by pressing the third finger placement portion 17 with the thumb T, and the bending operation lever 15 becomes a pressed-down state when an operation force amount F1 is applied to the lower side when viewed toward the paper surface which is the distal end side of the operation portion 3.

In this state, the operation portion 3 is grasped by the user by applying a force amount of a holding force F2 by the index finger IF to the first finger placement portion 13, applying a force amount of a holding force F3 by the middle finger MF to the second finger placement portion 14, and applying a force amount of a pressing force F4 by which the palm P presses a back surface of the grasping portion 12.

The holding force F2 applied to the first finger placement portion 13 is a force amount slightly downward from a front surface toward a back surface of the bending operation portion 11 (in oblique direction toward distal end side of operation portion 3). The holding force F3 applied to the second finger placement portion 14 is a force amount directed from the front surface to the back surface of the grasping portion 12. Further, the pressing force F4 of the palm P is a force amount slightly upward from the back surface toward the front surface of the grasping portion 12 (oblique direction toward proximal end side of operation portion 3).

The respective force amounts of the holding forces F2 and F3, and the pressing force F4 and the operation force amount F1 for holding the operation position of the bending operation lever 15 are substantially balanced, so that the user may stably grasp the operation portion 3 without shaking. In other words, the user may grasp the operation portion 3 of the endoscope 1 such that the respective force amounts of the holding forces F2 and F3 and the pressing force F4, and the operation force amount F1 are substantially balanced.

At this time, since the position of the end 13D on the distal end side of the first finger placement portion 13 is formed at a position separated to the front surface side from the end 13P on the proximal end side of the first finger placement portion 13 with the central axis X as a reference, the index finger IF may reliably press the first finger placement portion 13 and reliably apply the holding force F2 without slipping to the distal end side.

In other words, in the operation portion 3, the moment generated when the bending operation lever 15 is rotationally operated in one direction by the thumb T is canceled by the index finger IF pressing the first finger placement portion 13, the middle finger MF pressing the second finger placement portion 14, and the palm P pressing the grasping portion 12 from the back surface side.

Accordingly, when the bending operation lever 15 is rotationally operated in one direction by the thumb T, the user may stably grasp the operation portion 3 without shaking.

On the other hand, in the state illustrated in FIG. 7, in the operation portion 3, the operation position of the bending operation lever 15 is held in the state where the third finger placement portion 17 is pressed by the thumb T and pushed up toward the proximal end side of the operation portion 3. At this time, the bending operation lever 15 is in the state of being pushed up by the given operation force amount F1 in an obliquely upper left direction on the front surface side of the operation portion 3 from the upper side viewed toward the paper surface which is the proximal end side of the operation portion 3.

In this state, the operation portion 3 is grasped by the user by applying the force amount of the holding force F2 by the index finger IF to the first finger placement portion 13 and applying the force amount of the holding force F3 by the middle finger MF to the second finger placement unit 14.

The holding force F2 applied to the first finger placement portion 13 is a force amount obliquely downward from the front surface toward the back surface of the bending operation portion 11. Further, the holding force F3 applied to the second finger placement portion 14 is a force amount slightly downward from the front surface toward the back surface of the grasping portion 12.

Also in this case, since the holding forces F2 and F3, and the operation force amount F1 for holding the operation position of the bending operation lever 15 are substantially balanced, the user may stably grasp the operation portion 3 without shaking. In other words, the user may grasp the operation portion 3 of the endoscope 1 such that the respective force amounts of the holding forces F2 and F3, and the operation force amount F1 are substantially balanced.

In other words, in the operation portion 3, the moment generated when the bending operation lever 15 is rotationally operated in the other direction by the thumb T is canceled by the index finger IF pressing the first finger placement portion 13 and the middle finger MF pressing the second finger placement portion 14.

Therefore, even when the bending operation lever 15 is rotationally operated in the other direction by the thumb T, the user may stably grasp the operation portion 3 without shaking.

As described above, the endoscope 1 of the present embodiment is provided with the two recessed shapes of the first finger placement portions 13 for placing the index finger IF and the second finger placement portions 14 for placing the middle finger MF in the operation portion 3, and is configured in a shape such that the balance of force is balanced by canceling the operation force amount F1 generated in the operation portion 3 when the bending operation lever 15 is operated by the thumb T by each force amount (F2 and F3) applied from the index finger IF to the first finger placement portion 13 or from the middle finger MF to the second finger placement portion 14.

Further, since the force amount generated in the first finger placement portion 13 by placing the index finger IF thereon or in the second finger placement portion 14 by placing the middle finger MF thereon becomes a reaction force amount with respect to the force amount applied when operating the bending operation lever 15, the operation force amount of the bending operation lever 15 by the thumb T may be reduced, and thus slipping of the thumb T pressed against the third finger placement portion 17 of the bending operation lever 15 may be reduced.

Incidentally, when the endoscope 1 is, for example, for single-use (disposable), the mechanical configuration is required to be simple for manufacturing cost, and the mechanical structure in the bending portion 5 is simplified. However, in the single-use endoscope 1 in which the mechanical structure in the bending portion 5 is simplified, the operation force amount of the bending operation lever 15 tends to be heavy.

Therefore, the single-use endoscope 1 requires a large force amount for operating the bending operation lever 15 by the thumb T, the bending operation lever 15 of the bending portion 5 is heavy, and there is a possibility that finger-slip occurs.

Further, in the endoscope 1, in a case where a large force amount is required when operating the bending operation lever 15, the operation portion 3 grasped by the user may be shaken when operating the bending operation lever 15, and the position of the distal end of the insertion portion 2 may become difficult to be determined. Thus, stable photographing, treatment, and the like of the subject cannot be performed, and there is a concern that an examination and a procedure may be affected.

In order to approach these problems, the endoscope 1 according to the present embodiment is configured such that the bending operation lever 15 is comfortably operated without the operation portion 3 being shaken.

The endoscope 1 according to the present invention is not limited to an electronic endoscope and may be applicable to various endoscopes using a relay lens, an image fiber, and the like.

The invention described in the above embodiments and modifications is not limited to the embodiments and modifications described above, and various modifications may be made without departing from the scope of the invention in the implementation stage. Furthermore, the above-described embodiments and modifications include inventions at various stages, and various inventions may be extracted by appropriately combining a plurality of configuration requirements disclosed.

For example, even when some configuration requirements are deleted from all the configuration requirements described in the embodiments and the modifications, the configuration from which the configuration requirements are deleted may be extracted as the invention in a case where the described problems may be solved and the described effects may be obtained.

What is claimed is:

1. An endoscope operation portion comprising:
a grasping portion including a distal end connected to an elongated insertion portion having a bending portion that can be bent, the grasping portion having a longitudinal axis and being configured to be grasped by a palm of an operator;
a bending operation portion provided at a proximal end of the grasping portion and rotatably provided with a bending operation lever configured to bend the bending portion;
a first finger placement portion formed in the bending operation portion on one surface side of the operation portion and having a recessed shape configured to be placed with a first finger of the operator;
a second finger placement portion formed in the grasping portion on the one surface side of the operation portion and having a recessed shape configured to be placed with a second finger of the operator; and
a third finger placement portion provided on the bending operation lever arranged on another surface side opposite to the one surface side of the operation portion configured to be placed with a third finger of the operator, wherein
the first finger placement portion and the second finger placement portion are disposed at positions where at least a pressing force of the first finger placed on the first finger placement portion, a pressing force of the second finger placed on the second finger placement portion, and an operation force amount of the third finger placed on the third finger placement portion at the time of operating the bending operation lever are balanced.

2. The endoscope operation portion according to claim 1, wherein the first finger placement portion is formed at a position farther to the one surface side than the second finger placement portion with respect to the longitudinal axis.

3. The endoscope operation portion according to claim 1, wherein
the first finger placement portion includes a first end on a distal end side and a second end on a proximal end side, and
with respect to the longitudinal axis, a position of the first end on the distal end side is formed at a position farther to the one surface side than a position of the second end on the proximal end side.

4. The endoscope operation portion according to claim 1, wherein
the first finger placement portion includes a recessed shape having a first curvature and having an arc-shape in a cross-sectional direction, and
the second finger placement portion includes a recessed shape having a second curvature larger than the first curvature and having an arc-shape in a cross-sectional direction.

5. The endoscope operation portion according to claim 1, wherein the first finger of the operator is an index finger, the second finger is a middle finger, and the third finger is a thumb.

6. An endoscope comprising an endoscope operation portion, the endoscope operation portion including:
a grasping portion including a distal end connected to an elongated insertion portion having a bending portion that can be bent, the grasping portion having a longitudinal axis and being configured to be grasped by a palm of an operator;
a bending operation portion provided at a proximal end of the grasping portion and rotatably provided with a bending operation lever configured to bend the bending portion;
a first finger placement portion formed in the bending operation portion on one surface side of the operation portion and having a recessed shape configured to be placed with a first finger of the operator;
a second finger placement portion formed in the grasping portion on the one surface side of the operation portion and having a recessed shape configured to be placed with a second finger of the operator; and
a third finger placement portion provided on the bending operation lever arranged on another surface side opposite to the one surface side of the operation portion configured to be placed with a third finger of the operator, wherein
the first finger placement portion and the second finger placement portion are disposed at positions where at least a pressing force of the first finger placed on the first finger placement portion, a pressing force of the second finger placed on the second finger placement portion, and an operation force amount of the third finger placed on the third finger placement portion at the time of operating the bending operation lever are balanced.

7. An endoscope comprising:
a bending portion;
an operation portion configured to be grasped by an operator to operate the bending portion;
a bending operation lever provided freely movable in back and forth directions on a proximal end side of the operation portion and is configured to bend the bending portion;
a first finger placement portion having a recessed shape provided on a surface side opposite to a surface side provided with the bending operation lever of the operation portion and formed with a surface facing a distal end side configured to be placed with a first finger of the operator; and
a second finger placement portion having a recessed shape provided on a distal end side with respect to the first finger placement portion and formed with a surface facing a proximal end side configured to be placed with a second finger of the operator, wherein
a third finger placement portion is provided on the bending operation lever for placing a third finger of the operator, and
the first finger placement portion and the second finger placement portion are disposed at positions where at least a pressing force of the first finger placed on the first finger placement portion, a pressing force of the second finger placed on the second finger placement portion, and an operation force amount of the third finger placed on the third finger placement portion at the time of operating the bending operation lever are balanced.
